(19) United States
(12) Patent Application Publication
(10) Pub. No.: US 2014/0273133 A1
(43) Pub. Date: Sep. 18, 2014

(54) Ethanolic Fermentation with Immobilized Yeast

(71) Applicant: University of Idaho, Moscow, ID (US)

(72) Inventor: Tushar Jain, Nampa, ID (US)

(73) Assignee: UNIVERSITY OF IDAHO, Moscow, ID (US)

(21) Appl. No.: 14/209,521

(22) Filed: Mar. 13, 2014

Related U.S. Application Data
(60) Provisional application No. 61/781,449, filed on Mar. 14, 2013.

(51) Int. Cl. C12P 7/06; C12N 11/10

(52) U.S. Cl. CPC C12P 7/06; C12N 11/10
USPC 435/161

(57) ABSTRACT

Methods of fermentation utilizing immobilized yeast, wherein growth of the yeast during fermentation is inhibited, provide advantages over methods in which yeast are permitted to grow during fermentation.

This application claims priority from pending U.S. Provisional Patent Application Ser. No. 61/781,4419, filed Mar. 14, 2013.

### FIELD OF THE INVENTION

This invention pertains to the field of fermentation processes utilizing yeast to produce alcohols such as ethanol and butanol.

### BACKGROUND OF THE INVENTION

Yeasts are unicellular eukaryotic microorganisms presently classified as fungi. They have been used for thousands of years for their ability to convert carbohydrates to carbon dioxide and alcohol, a process known as fermentation. The fermentation process is used extensively in the production of alcoholic beverages such as beer and wine and, in recent years, has been utilized in the manufacture of ethanol fuel.

The United States and Brazil produce about 70% of the world's ethanol. Most commonly, in the United States, a batch fermentation process is utilized in Which the yeast, once used is not collected for reuse. In this process, a fermentation tank is filled with a batch containing yeast and a feedstock that is allowed to ferment to completion, after which the tank is drained in order to recover the ethanol product and the yeast, the ethanol and yeast are separated, and the tank is refilled with a new batch for another round of fermentation. Batch fermentation processes generally require about 36 to 48 hours to completely ferment the feedstock.

In Brazil, a common fermentation process is a semi-continuous fermentation process known as the Melle-Boinot process in which increased yeast concentrations are utilized, typically up to 30 w/w of the batch, and yeast are collected following fermentation and reused. for the next fermentation. With the Melle-Boinot process, fermentation times are greatly reduced, from the typical 36 to 48 hours in the United States to about 6 to 8 hours. One disadvantage of the Melle-Boinot process is that it produces large quantities of foam due to the high surface tension between yeast particles, which necessitates the use of anti-foaming agents. Another disadvantage of the Melle-Boinot fermentation system is that the yeast must be separated from the ethanol that is produced b the fermentation process. Most commonly, the yeast is separated from the ethanol liquid by centrifugation.

In order to alleviate problems associated with separation of yeast from ethanol, methods of encapsulating yeast have been developed. Because surface tension is greatly reduced when yeast are encapsulated, foaming is minimal using encapsulated yeast. Hill, U.S. Pat. No. 5,070,019, discloses a method of encapsulating and immobilizing yeast in calcium alginate beads. According to Hill, the yeast-containing calcium alginate beads are made by adding dropwise a 1 to 4% solution of sodium alginate m which yeast are suspended to a precipitation bath containing a CaCl₂ solution. This results in the production of calcium alginate beads in which yeast are encapsulated. The beads have diameters of 1 to 6 mm, a biomass content of 15 to 80%, an alginate content of 80 to 15%, and a residual moisture content of 5 to 20%. The calcium alginate/yeast beads are disclosed to be useful in the production of alcoholic beverages.

Juong, U.S. Pat. No. 4,996,150, discloses that prior art methods of making calcium alginate beads were problematic because they did not have a practical means for obtaining beads of uniform size. Juong discloses that bead size is an important consideration because yeast cells within the interior of large beads cannot proliferate and contribute to the fermentation process because only yeast cells located close to the surface of the beads are able to obtain nutrients necessary for the growth of yeast that is required for fermentation. In order to solve this problem, Juong discloses the incorporation of a biocatalytic system containing yeast within a homogenous mixture of calcium alginate and a cationic polymer such as polyethyleneimine (PEI). This process is disclosed to result in the production of beads of uniform size that can accommodate yeast expansion due to yeast growth.

Lommi, U.S. Pat. No. 5,079,011, discloses a matrix upon which yeast are immobilized. Lommi discloses that, during the fermentation process, yeast within the matrix are constantly growing and are maintained by the supply of fermentable sugars in the medium which acts as a nutrient source. During the fermentation process, the numbers of yeast cells are maintained at a constant level by balancing the increased numbers of yeast cells that are produced by growth and the leaching of dead yeast cells.

Juong, Annals New York Academy of Sciences, pages 271-282 (1990), addresses the problem of lack of expansibility of yeast carrier systems. This lack of expansibility restricts the growth of yeast within the system, with deleterious effects. in order to solve this problem. Juong discloses a closed bioreactor system in which yeast immobilized in PEI-alginate beads continually proliferate reports that yeast immobilized within the beads continually proliferate and that this growth results in a 1000% increase in volume of the heads during their useful lifetime.

Yeast for fermentation have been encapsulated in materials other than calcium alginate. Harder, U.S. Pat. No, 5,405,764, discloses the encapsulation of microorganisms such as yeast in gelatin or chitosan. Hsu, U.S. Pat. No. 4,659,662, discloses that yeast for fermentation may be encapsulated in beads of sulfated polysaccharides such as carrageenan, filrcellaran and cellulose sulfate, polyacrylanride, sodium alginate, polyvinyl alcohol, cellulose succinate, casein succinate, polymerized acrylates and methacrylates, gelatin, acryloyl polymers, carboxymethyl cellulose, and light-or radiation-curable resins. Tan et al, Pharmaceutics, 3:731-744 (2011), discloses yeast for fermentation encapsulated in gellan gum.

### DESCRIPTION OF THE INVENTION

It has been discovered that inhibiting or eliminating growth in yeast that are immobilized, such as by being encapsulated in beads, provides many unexpected advantageous properties when utilizing. the immobilized yeast for fermentation. When yeast are immobilized by encapsulation in beads, growth of the yeast is preferably inhibited or eliminated by IS encapsulating the yeast in beads in a sufficiently high concentration so that reproduction of the yeast within the beads is inhibited due to space constraints and reduction of diffusion of oxygen and nutrients within the beads.

As with prior art fermentation processes using immobilized yeast, such as yeast encapsulated in heads, fermentation performed using the immobilized yeast, such as yeast immobilized in heads, described in this application provides benefits of reduced foam production compared with fermentation using free yeast. Also, as with such prior art fermentation processes, the immobilized yeast of this application are easily separated from a fermented solution by the use of mesh in which beads are readily entrapped.

In addition to the benefits obtained by utilizing immobilized yeast, such as yeast impregnated beads, for fermentation rather than free yeast, other unexpected benefits are obtained by fermentation utilizing the yeast-impregnated beads of the present application.

Fermentation processes utilizing the immobilized yeast, such as yeast immobilized in beads, of the present application produce an increase in yield, estimated to be about 5% higher than obtainable from present fermentation methods. Yields of 95% have been obtained using the fermentation process of the present application. Because reproduction of yeast cells with the method of the present application is reduced or eliminated, there is little or no change in strain quality of yeast over time and, consequently, the yeast utilized in the method remain the same, which eliminates the necessity of present fermentation methods to intermittently remove the yeast and replace the yeast with new yeast having the original characteristics. With the method of the present application, because the yeast population does not reproduce or reproduces minimally, the yeast population does not mutate or mutates minimally and, therefore, the same yeast may be used and reused many times without a change in the characteristics of the final fermented product. Also, because reproduction of the yeast is reduced or eliminated, there is little or no need to provide nutrients and oxygen for the yeast, which would be utilized for reproduction. Moreover, reproduce of the yeast is reduced, the longevity of the yeast is increased, allowing the yeast to be used .for many fermentation batches, even as much as 50 batches or more, while retaining vitality. The immobilized yeast of the application have been used in 50 consecutive fermentation batches while maintaining about 95% vitality after the 50 batches. Additionally, heat produced due to reproduction of yeast is reduced or eliminated, thus reducing or eliminating, the necessity of removing the excess heat produced by present fermentation methods. Moreover, fermentation processes utilizing the beads of the present application produce alcohol such as ethanol in markedly reduced time compared with prior art fermentation methods. Significantly, this fast fermentation process according to the present method occurs with very little foam produced, which is advantageous because the present method eliminates the need for anti-foaming agents or additional steps to remove excess foam.

The prior art did not recognize that the restriction of the growth of yeast encapsulated within heads is a result-effective variable. Rather, the dogma of the prior art is that yeast must be permitted to grow throughout the fermentation process. However, the present application proceeds directly contrary to the accepted. wisdom the art and restricts or eliminates growth of immobilized yeast, such as yeast encapsulated within beads. By doing so, the above mentioned unexpected advantageous benefits are obtained.

In accordance with the present application, yeast are immobilized, such as on a carrier as described in Lommi, U.S. Pat. No. 5, 79,011, in a semi-permeable membrane as described in Hsu, U.S. Pat. No. 4,659,662, or within beads. In one preferred embodiment, beads for fermentation of carbohydrates are made by encapsulating yeast within a bead, such as a calcium alginate bead, at a concentration that is sufficiently high to inhibit or eliminate yeast reproduction and growth due to limited space availability within the beads. Because the yeast cells are unable to reproduce, sugars in the medium are utilized predominately or completely for fermentation and conversion into alcohol, such as ethanol, rather than as a nutrient for the yeast. In addition to the benefits mentioned above, the high yeast concentrations and nutrient lean environment that is obtained in the beads of this application inhibit the reproduction of microorganisms other than yeast and, therefore, decrease the formation of undesirable byproducts, such as aldehydes and ketones, by these other microorganisms, adding to the efficiency of the process.

In a less preferred embodiment of the present application, yeast are immobilized, such as on a carrier or within beads, and either or both of nutrients and oxygen is not supplied to the yeast during fermentation, or nutrients and/or oxygen is supplied in an amount that results in a reduction in growth that would be obtained if sufficient nutrients and oxygen were supplied to the yeast during fermentation. If either nutrients or sugar are not supplied to yeast during fermentation, growth of the yeast will be inhibited or eliminated during fermentation, which will provide the unexpected advantages of this application.

In the present application, the invention is described and exemplified utilizing calcium alginate beads in which yeast are encapsulated. However, yeast encapsulated in beads, and particularly calcium alginate beads, are merely exemplary and the invention is applicable to any immobilized yeast or to any beads in which yeast may be encapsulated while retaining the ability to fertilize a substrate in which the yeast or beads are situated. Thus, as disclosed in the prior art, examples of beads in which the methods of the present application are suitable include, but are not limited to, beads composed of gelatin, chitosan, sulfated polysaccharides such as carrageenan, furcellaran and cellulose sulfate, polyacrylamide, sodium alginate, polyvinyl alcohol, cellulose succinate, casein succinate. polymerized acrylates and methactylates, gelatin, acryloyl polymers, carboxymethyl cellulose, light-or radiation-curable resins, or gellan gum. Moreover, the invention is applicable to yeast that are immobilized by other than in beads, such as upon 3 matrix.

In the present application, the method of the invention is described and exemplified utilizing yeast, and particularly Saccharomyces cerevisiae (Baker's yeast), as the fertilizing microorganism. However, this organism is merely exemplary and the invention is applicable to any microorganism that is capable of being encapsulated and of fertilizing a substrate while encapsulated. Thus, the invention is applicable to other yeasts, such as those that have been used for the production of potable alcoholic beverages, including other species of Saccharomyces, such as S. pastorianus, S. boyanus, and S. boulardii, and yeast species other than Saccharomyces, such as those of the genera Brettanomyces or Dekkera. In addition to organisms like yeast, the invention is also applicable to bacterial organisms that produce useful products by fermentation, such as Clostridium spp., such as for the production of acetone, n-Butanol, and ethanol from starch, or Lactobacillus spp., such as for the production of food products such as yogurt, cheese, sauerkraut, pickles, beer, wine, cider, or kimchi.

Methods for making beads of materials such as calcium alginate or of materials other than calcium alginate such as those listed above, and for encapsulating, yeast with the beads, are known in the art. For example if using calcium alginate beads as an encapsulation matrix, the beads may be made by suspending yeast in an aqueous solution of sodium alginate, such as described in Hill, U.S. Pat. No. 5,070,019. The concentration of sodium alginate in the solution is generally in the range of 0.1 to 5 wt. %, although higher or lower concentrations may be utilized if desired. Yeast particles are suspended in the solution at a concentration sufficient to provide the desired ratio as disclosed in the next paragraph. Generally, a concentration of yeast between 10 to 35 wt %, such as 20%, is utilized. If desired, concentrations of yeast higher than 35 wt %, such as between 35 and 50 wt %, may be utilized. Because the suspension is added dropwise to a precipitation bath, the upper concentration of yeast is only limited to that which will permit the formation of discrete uniform drops of the suspension. The suspension containing the yeast in the sodium alginate is then added dropwise to a precipitation bath containing calcium chloride (CaCl₂) in solution. Generally, the concentration of CaCl₂ in the solution is between 0.5 to 20.0 wt %, although higher or lower concentrations may be utilized if desired.

Prior art methods of yeast encapsulation in alginate utilize a yeast biomass content of heads in the range of 15 to 80 wt. % and an alginate content of 80 to 15 wt. %. See Hill, U.S. Pat. No. 5,070,019. Thus, the ratio of yeast:alginate is disclosed to be between 15:80 and 80:15. Applicant has discovered that the relative concentration of yeast and alginate at levels higher than 80:15 (5:33:1), and preferably at levels higher than 10:1, more preferably at levels higher than 20:1, even more preferably at levels higher than 25:1, and most preferably at levels higher than 30:1, such as 40:1 or higher, is a result-effective variable that provides the advantages mentioned above. These ratios pertaining to alginate are likewise applicable to beads made of materials other than alginate, such as carrageenan, furcellaran and cellulose sulfate, polyacrylamide, sodium alginate, polyvinyl alcohol, cellulose succinate, casein succinate, polymerized acrylates and methacrylates, gelatin, acryloyl polymers, carboxymethyl cellulose, chitosan gellan gum, and light-or radiation-curable resins.

Prior art methods of yeast encapsulation in alginate utilize a ,feastlsoclium alginate slurry in which the concentration of sodium alginate varies from 0.5 wt % to 5. 0 wt % , and the concentration of yeast in the slurry is about 10 times that of the sodium alginate on a dry weight basis, that is between 5 and 50 wt in accordance with the method of preparation of alginate/yeast beads of the present application, the concentration of yeast in the sodium alginate slurry is that which is sufficient to restrict growth of the yeast within calcium alginate beads made by adding the slurry dropwise to a precipitation bath containing calcium chloride (CaCl₂) in solution. Thus, the concentration of yeast in the sodium al mate slurry (w/w) is at least 15 times that of the concentration of sodium alginate in the slurry (w/w), preferably at least 20 times that of sodium alginate, more preferably at least 25 times that of sodium alginate, even more preferably at least 30 times that of sodium alginate, and most preferably at least 40 times that of sodium alginate in the slurry.

Thus, in the calcium alginate beads of the present application, the percentage dry matter that is yeast is greater than 85%, preferably at least 91%, more preferably at least 95%, and most preferably at least 96 8%, such as 97.5% or higher. For beads made of materials other than calcium alginate, the percentage dry matter that is yeast is similar to that recited above but may differ slightly based on different molecular weights of various materials. Thus, for materials other than calcium alginate the percentage dry matter that is yeast is greater than 80%, preferably at least 85%, more preferably at least 90%, even more preferably at least 95%, and most preferably at least 96.8%, such as 97.5% or higher.

Beads such as calcium alginate beads made according to this method generally have a diameter of 1 to 10 mm, but may be smaller than 1 mm or larger than 10 mm. The beads may of any shape, such as in the shape of spheres discs, or cubes. If desired, the beads may be hardened, such ashy allowing calcium alginate heads to remain in a CaCl₂ solution until they reach a desired degree of hardness. In a preferred embodiment, calcium alginate beads are hardened in a CaCl₂ solution for a period of between 1 minute and 2 hours, preferably between 2 and 1 hour, more preferably between 5 and 30, even more preferably between 8 and 20 minutes, with a preferred residence time in the CaCl₂ solution of about 10 to 15 minutes, such as 12 minutes. The beads may be dried if desired. If dried, a preferable residual moisture content is between 0.1% and 10%, with a more preferred residual moisture content between 0.25 and 5%, a most preferred residual moisture content between 0.5% and 3%, for example a moisture content of around 1%. Once dried, the beads may be stored for long periods of time. In the dried beads, the biomass is preferably in the range of 90 to 99 wt %, the alginate content is preferably in the range about 1 to 10 wt %, and the residual moisture is preferably in the range of 0.25 to 5 wt %.

The beads, such as calcium alginate beads made as described above, may be washed, either after drying, or before drying, or after the beads are formed and without a subsequent drying step. Washing is done by rinsing, bathing, immersing, shaking, or stirring in an amount of water sufficient to wash the beads. The washing may be under running water or in a container. Generally, the amount of water used to wash the beads involves the use of a 10 to 300 fold excess of water or higher, based on weight of the beads.

The beads, as described above, may be utilized for the fermentation of any feedstock that is fermentable by yeast, such as wort, fruit juice, betty juice, sugar syrup, starch syrup, a hydrolysate of a plant material, or any substrate that is used in the production of beer, wine, or liquor, including ethanol and butanol.

In a less preferred embodiment of this application, growth of immobilized yeast during fermentation is inhibited or eliminated by other than by the dense packing of the yeast within beads as described above. In the case when the concentration of yeast is less than described above, such as when the relative concentration of yeast and alginate is not higher than 80:15 (5.33:1), or when the percentage dry matter that is yeast is not greater than 85% in the case of calcium alginate beads or not greater than 80% in the case of beads other than calcium alginate, or when the yeast are immobilized by other than encapsulation in beads, such as b immobilization on a matrix, growth of yeast during fermentation may be inhibited or eliminated by restricting the supply of nutrients and/or supply of oxygen to the yeast during fermentation. For example, fermentation may be performed under anaerobic conditions.

Such methods are less preferred because, due to the fact that a lower concentration of yeast n beads is utilized, the fermentation that is obtained is not as rapid as that obtained using, the high concentrations of yeast as described above. However, other advantages of the present invention, such as increase in yield, lack of buildup of heat during fermentation, ability to reuse the yeast for many fermentation cycles without change in characteristics of the yeast over time, and reduced foam production, are obtained.

Growth, which is considered to be equivalent to reproduction, of yeast can be determined by measuring, such as by weighing, the biomass of dried yeast before and after fermentation. For purposes of this application, growth of yeast is considered to be inhibited if biomass increases less than 5% during. fermentation of a batch. Preferably, growth is limited to an increase in biomass of less than 1% per batch.

The invention is further illustrated in the following non-limiting examples. In the following examples, the invention is illustrated utilizing yeast encapsulated in calcium alginate beads. It is understood that calcium alginate beads are merely illustrative of the invention and beads, and immobilization means, other than yeast may be utilized within the scope of the present invention.

### EXAMPLE 1

### Preparation of Yeast Encapsulated in Beads

Dehydrated Baker's east was obtained from a grocery store. Sodium alginate derived from Macrocystis pyrifera was obtained from Sigma Chemical Co., St. Louis, Mo. The Baker's yeast was rehydrated with water to make 6 kg of 33% yeast slum which was maintained at 4° C. A 4 kg dispersion of 50 gm sodium alginate was prepared using a blender. The yeast slurry and sodium alginate solution were mixed to make a yeast-sodium alginate dispersion having the concentrations shown in Table 1.

TABLE 1

| | |
|---|---|
| Yeast (Dry weight) | 2.0 kg |
| Sodium Alginate | 0.05 kg |
| Water | 7.95 kg |

Five (5) liters of a 2% CaCl₂ solution was prepared in water. The yeast-sodium alginate dispersion was added dropwise from a bead-maker of 70 syringes and using a peristaltic pump. The beads produced were removed from solution after 10 minutes and were dried to reduce moisture to about 1 wt %. The dried beads were noted to shrink to about one-third of their size prior to drying.

### EXAMPLE 2

### Fermentation using the Beads of Example 1

Higher concentrated sugar solution obtained from corn syrup was fermented with rehydrated beads of Example 1 under anaerobic conditions. The corn syrup was obtained from a grocery store. The syrup was diluted to sugar concentration of 35° brix. 30 gm of dried beads were rehydrated by soaking them in water for 30 minutes. 100 gm of corn syrup was circulated through the rehydrated beads.

Carbon dioxide release due to fermentation was measured. The brix number was reduced to 15° brix in two hours. The beads were recovered from the solution and the solution was distilled to remove the alcohol.

### EXAMPLE 3

### Repeated Fermentation Processes

The residual solution remaining after removal of the alcohol in Example 2 was made to 35 brix by adding additional water and corn syrup. The procedure of Example 2 was then repeated.

Repeated cycles of alcohol removal, bringing the residual solution to 35° brix and fermentation were performed without adding more yeast. The cycle was repeated 50 times. In each repetition, about 20% sugar was converted to ethanol and was distilled and about 10% alcohol by volume was recovered after every hatch. After 50 cycles, the yeast were dried and the biomass of the yeast was determined by weight. It was determined that the biomass of the yeast had increased by only 0.6% during the 50 cycles.

### EXAMPLE 4

### Fermentation Process

Utilizing the beads of Example 1, 50 batches of 22° brix sugar solution was fermented in less than 6 hours per batch, using the same beads throughout the 50 batches.

### EXAMPLE 5

### Fermentation Process

Utilizing the beads of Example 1, a 12° brix sugar solution was fermented in less than 3 hours. Using the same beads, fermentation cycles were repeated 50 times. Fermentation times were almost constant throughout the 50 cycles, varying from about 130 minutes to about 160 minutes, indicating that viable cell counts were stable throughout the 50 batches.

Various modifications of the above described invention will be evident to those skilled in the art. It is intended that such modifications are included within the scope of the following claims.

1. A method for fermenting a carbohydrate feedstock comprising combining the feedstock with immobilized yeast and permitting the yeast to ferment the feedstock, wherein growth of the yeast during the fermentation is inhibited or eliminated.

2. The method of claim I wherein the growth of the yeast is less than 5% per fermentation batch.

3. The method of claim 1 wherein the growth of the yeast is less than 1% per fermentation batch.

4. The method of claim 1 wherein the growth of the east during the fermentation is inhibited or eliminated by restricting the supply of nutrients or oxygen to the yeast during the fermentation.

5. The method of claim 4 wherein the fermentation is performed under anaerobic conditions.

6. The method of claim 1 wherein the yeast are immobilized in beads.

7. The method of claim 6 wherein the beads are calcium alginate.

8. The method of claim 7 wherein the relative concentration of yeast and alginate is higher than 80:15.

9. The method of claim 8 wherein the relative concentration is at least 10:1.

10. The method of claim 9 wherein the relative concentration is at least 20:1.

11. The method of claim 10 wherein the relative concentration is at least 25:1.

12. The method of claim 6 wherein the beads have a dry matter content that is greater than 85% yeast

13. The method of claim 12 wherein the dry matter content is at least 91% yeast.

14. The method of claim 13 wherein the dry matter content is at least 95% yeast.

15. The method of claim 6 wherein the heads are subjected to a drying step.

16. The method of claim 15 wherein the dried beads have a residual moisture content between 0.1% and 10%.

17. The method of claim 16 wherein the dried beads have a biomass that is between 90% and 99% of the weight of the dried beads.
